(19) United States
(12) Patent Application Publication
(10) Pub. No.: US 2017/0304260 A1
(43) Pub. Date: Oct. 26, 2017

(54) ALTRENOGEST FORMULATION AND USES THEREOF FOR ESTRUS SYNCHRONISATION IN ANIMALS

(71) Applicant: Maamar Achacha, Mont St-Hilaire (CA)

(72) Inventor: MAAMAR ACHACHA, MONT ST-HILAIRE (CA)

(21) Appl. No.: 15/517,989

(22) PCT Filed: Oct. 8, 2015
(86) PCT No.: PCT/CA2015/051019

§ 371 Date: Apr. 10, 2017

Related U.S. Application Data
(60) Provisional application No. 62/061,422, filed on Oct. 8, 2014.

(51) Int. Cl. A61K 31/355; A61K 47/34; A61K 31/575; A61K 9/16; C07J 1/00; A61K 47/00; A61K 9/00

(52) U.S. Cl. CPC A61K 31/355; A61K 9/1658; A61K 47/34; C07J 1/00; A61K 31/575; A61K 9/00; A61K 47/00

(57) ABSTRACT

The invention relates to a microsphere composition comprising (a) a microsphere material; and (b) a lipidic liquid formulation comprising (i) an effective amount of a progestogen, and (ii) vitamin E or an analog of vitamin E, incorporated within said microsphere material, into said microspheres. The composition may be used to manage and control the synchronization of estrus in animals.

### CROSS REFERENCE TO RELATED APPLICATIONS

This application claims the benefit of U.S. provisional application Ser. No. 62/061,422 filed on Oct. 8, 2014, which is incorporated herein by reference in its entirety.

### TECHNICAL FIELD

The present invention generally relates to the field of artificial insemination and embryo transfer in animals, and more specifically to the management and control of estrus suppression/synchronisation in animals.

### BACKGROUND ART

In animal production, “heat synchronization” or “estrus synchronization” traditionally refers to artificially synchronizing the estrus cycle in a group of females by using hormonal manipulation.

A major goal of commercial swine production is to maximize reproductive efficiency, especially among gilts. Increased reproductive efficiency offers producers substantial opportunities to reduce production costs and enhance profitability. Efforts are being made to increase reproductive efficiency by breeding gilts at earlier ages, synchronizing estrus among the gilts, impregnating gilts using artificial insemination (AI) and increasing the litter size and increasing the birth and weaning weight of the litters. Gilts can be bred at earlier ages by chemically inducing puberty. There are no products currently effective at regulating estrus once gilts have started to cycle so that estrus synchronization during the more productive second or third estrus cycles is presently not possible. The most common alternative management technique is to synchronize estrus in groups of gilts by first mating the gilts and then administering Prostaglandin F2α (PGF2α) to induce “synchronous abortion” as soon as two to three weeks, and up to eight to ten weeks after the end of the mating period. This method of synchronizing estrus is presently used in some large swine operations, but it requires extra boars and extra boar housing. In addition, aborted concept uses are unsanitary and may cause gilts to develop endometriosis after aborting.

Altrenogest or 17-allyltrenbolone is a progesterone synthesis agonist, structurally and pharmacologically close to the latter. Like all steroids, its liposolubility allows it to penetrate the target cells, where it bonds to specific intracellular receptors. It is used in veterinary medicine, in particular in sows and mares, for zootechnical purposes, orally, for estrus synchronization. Its most significant effects are the progestomimetic and anti-gonadotropin effects. Altrenogest-based oral formulations (solutions) are marketed under the trade names REGUMATE® and ALTRESYN®.

There is thus a need for improved compositions and methods for controlling the reproductive pathway for swine, especially gilts, for example methods that do not involve daily injections or the use of non-biodegradable injected implants that need to be removed at the end of treatment.

The present description refers to a number of documents, the content of which is herein incorporated by reference in their entirety.

### SUMMARY OF THE INVENTION

The present invention relates to the following items 1 to 39:

1. A biodegradable microsphere composition comprising (a) a biodegradable microsphere material; and (b) a lipidic liquid formulation comprising (i) an effective amount of a progestogen, and (ii) vitamin E or an analog of vitamin E, incorporated within said biodegradable material, into said microspheres.
2. The biodegradable microsphere composition according to item 1, wherein the progestogen is altrenogest:

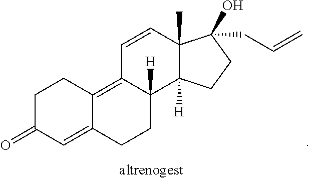

3. The biodegradable microsphere composition of item 1 or 2, comprising an analog of vitamin E.
4. The biodegradable microsphere composition of item 3, wherein the analog of vitamin E is tocopheryl acetate.
5. The biodegradable microsphere composition of any one of items 1 to 4, comprising one or more antioxidants or preservative agents.
6. The biodegradable microsphere composition of item 5, wherein the antioxidant or preservative agent(s) is (are) sorbic acid, butylated hydroxyanisole and/or butylated hydroxytoluene (BHT)
7. The biodegradable microsphere composition of item 5 or 6, wherein the antioxidant or preservative agent(s) is (are) present in the formulation at a concentration of about 0.001 to 1%.
8. The biodegradable microsphere composition of item 7, wherein the antioxidant or preservative agent(s) is (are) present at a concentration of about 0.01% to 0.02%.
9. The biodegradable microsphere composition of any one of items 5 to 8, comprising about 0.001% to 0.01% of butylated hydroxyanisole, about 0.001% to 0.01% of butylated hydroxytoluene, and about 0.01 to 0.02% of sorbic acid.
10. The biodegradable microsphere composition of any one of items 1 to 9, further comprising an alcohol.
11. The biodegradable microsphere composition of item 10, wherein the alcohol is butyl alcohol.
12. The biodegradable microsphere composition of item 10 or 11, wherein the alcohol is present at a concentration of about 0.5 to 2%.
13. The biodegradable microsphere composition of item 12, wherein the alcohol is present at a concentration at about 1%.
14. The biodegradable microsphere composition of any one of items 1 to 13, comprising an oil.
15. The biodegradable microsphere composition of item 14, wherein the oil is soybean oil, sunflower oil, or primrose oil.
16. The biodegradable microsphere composition of item 14 or 15, wherein the oil is present at a concentration of approximately 85% to 90%.
17. The biodegradable microsphere composition of any one of items 1 to 16, wherein the progestogen is present at a concentration of about 0.01 to 1%.
18. The biodegradable microsphere composition of item 17, wherein the progestogen is present at a concentration of about 0.2%.
19. The biodegradable microsphere composition of any one of items 1 to 18, wherein the vitamin E or the vitamin E analog is present at a concentration of approximately 0.01 to 1%.
20. The biodegradable microsphere composition of item 19, wherein the vitamin E or vitamin E analog is present in a concentration of about 0.05%.
21. The biodegradable microsphere composition of any one of items 1 to 20, wherein the biodegradable microsphere material comprises a biodegradable polymer, a polysaccharide, a protein, or any mixtures thereof.
22. The biodegradable microsphere composition of item 21, wherein the biodegradable microsphere material comprises a protein.
23. The biodegradable microsphere composition of item 22, wherein the biodegradable microsphere material comprises sodium caseinate.
24. The biodegradable microsphere composition of any one of items 21 to 23, wherein the biodegradable microsphere material comprises xantham gum, chitosan and/or maltodextrin.
25. The biodegradable microsphere composition of any one of items 1 to 24, which is in liquid form.
26. The biodegradable microsphere composition of item 25, wherein the biodegradable microspheres are in an oil or aqueous solution.
27. The biodegradable microsphere composition of any one of items 1 to 24, which is in solid form.
28. The biodegradable microsphere composition of item 25, wherein the biodegradable microspheres are in a dry powder.
29. The biodegradable microsphere composition of any one of items 1 to 28, wherein the biodegradable microsphere composition is in a capsule, a transdermal patch or food.
30. A method for improving the regularity of the appearance of estrus in an animal, the method comprising the administration of the biodegradable microsphere composition of any one of items 1 to 29 to an animal.
31. A method for synchronizing time of insemination in an animal, the method comprising the administration of the biodegradable microsphere composition of any one of items 1 to 29 to an animal.
32. The method of item 31, wherein the insemination is an artificial insemination.
33. The method of any one of items 30 to 32, wherein the animal is a sow or gilt.
34. Use of the biodegradable microsphere composition of any one of items 1 to 29 to improve the regularity of the appearance of estrus in an animal.
35. Use of the biodegradable microsphere composition of any one of items 1 to 29 for the preparation of a medicament to improve the regularity of the appearance of estrus in an animal.
36. Use of the biodegradable microsphere composition of any one of items 1 to 29 for synchronizing time of insemination in an animal.
37. Use of the biodegradable microsphere composition of any one of items 1 to 29 for the preparation of a medicament for synchronizing time of insemination in an animal.
38. The use of item 36 or 37, wherein the insemination is an artificial insemination.
39. The use of any one of items 34 to 38, wherein the animal is a sow or gilt.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the rate of synchronized sows in the nine farms studied. LA=LAS; SF=St-Francoise; SO=St-Octave; Ge=Genetiporc; SA=Ste-Anne; CB=CB; Na=Nathali; Jo=Jolivoir; SH=St-Honoré. Left bars=% synchronisation; right bars=% gestation.

FIG. 2 shows the percentages of responding sows following treatment with 2 mg/ml or 4 mg/ml of ALT. Left bars=% synchronisation; right bars=% gestation.

FIG. 3 shows the number of sows in estrus and in farrowing at day 3 (left bars), day 4 (middle bars) and day 5 (right bars) post-treatment with 2 mg/ml or 4 mg/ml of ALT for 10 or 15 days. L1-10=Livestock 1, 10-day treatment; L1-15=Livestock 1, 15-day treatment; L2-10=Livestock 2, 10-day treatment and L2-15=Livestock 1, 15-day treatment.

FIG. 4 shows the percentages of sows in estrus (left bars) and in farrowing (right bars) treated with 2 mg/ml or 4 mg/ml of ALT for 10 or 15 days. L1-10=Livestock 1, 10-day treatment; L1-15=Livestock 1, 15-day treatment; L2-10=Livestock 2, 10-day treatment and L2-15=Livestock 1, 15-day treatment.

FIG. 5 shows the percentage of farrowing sows following treatment with 2 mg/ml or 4 mg/ml of ALT, or with 2.2 mg/ml of Regumate® (control 1 and control 2); and

FIG. 6 shows the number of newborn piglets, total (left bars) and living (right bars) after treatment with 2 mg/ml or 4 mg/ml of ALT, or with 2.2 mg/ml of Regumate® (control 1 and control 2).

### DISCLOSURE OF INVENTION

The results presented herein provides evidence that treatment with a novel altrenogest formulation (herein referred to as ALTERVITAMIN or ALT) has an efficacy that is at least equivalent or even superior (at least in terms of farrowing rates) to conventional altrenogest-based treatment with Regumate® (oil formulation). The rate of heat synchronization obtained in the studies using ALTERVITAMIN was 90%, which is higher than that obtained in the majority of studies using progestogens. Without wishing to be bound by theory, this high rate is believed to be explained, at least in part, by the coating of the formulation with biodegradable microspheres/microcapsules, which may make it possible to improve the bioavailability and/or chemical stability of the ingredients with respect to certain animal digestive enzymes and lipid oxidation, and also by the presence of vitamin E that may have an indirect effect on the bioavailability of prostaglandins on the one hand and endogenous progesterone on the other hand. The microspheres had good rehydration properties, low surface oil and low oxidation during storage, and may be conveniently formulated in liquid (e.g., oil, aqueous) or solid form (e.g., dry powder), in contrast to the Regumate® oil formulation.

The present invention relates to a lipidic liquid formulation comprising: a progestogen, such as altrenogest, vitamin E (or a derivative of vitamin E) and optionally one or more excipients.

The present invention also relates to a microsphere composition comprising a microsphere-forming material, such as a protein, polymer, or polysaccharide, and the lipidic liquid formulation defined herein incorporated within said microsphere-forming material, into said microspheres.

The present invention also relates to a biodegradable microsphere composition comprising a biodegradable microsphere-forming material, such as a biodegradable protein, polymer, or polysaccharide, and the lipidic liquid formulation defined herein incorporated within said biodegradable microsphere-forming material, into said biodegradable microspheres.

The size of these microspheres may be between about 0.03 and 1000 micrometers (μM) thereby forming a colloidal dispersion in water. The term “microsphere” as used herein essentially means structures having a size range of between about 0.03 and 1000 μM. In embodiments, the microspheres have a size of between about 0.1 and 1000 μM, between about 1 and 500 μM, between about 1 and 100 μM, or between about 1 and 10 μM. In another embodiment, the microspheres have a mean size of about 1 μM.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

The term progestogen (or progestin) refers to a molecule of natural or synthetic origin having a progestin activity and having anti-estrogen and anti-gonadotropic properties, such as progesterone, progesterone derivatives or 17-hydroxyprogesterone, or the derivatives of testosterone or nortestosterone, to which substituted radicals give a progestin activity (altrenogest).

In one particular embodiment, the progestogen is altrenogest. Altrenogest is a synthetic progestogen having the following structure:

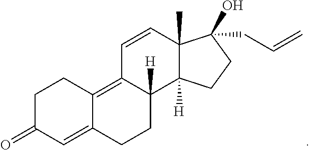

The progestogen, preferably altrenogest, is present in the composition at a concentration sufficient to bring about a progestin activity in an animal. In one particular embodiment, the progestogen, preferably the altrenogest, is present in the composition at a concentration of approximately 0.001 to 5% (w/vol), or at a concentration of approximately 0.01 to 1%, for example a concentration of approximately 0.2 to 0.3%.

The term “vitamin E derivative” refers to a molecule having a structure and an activity comparable to those of vitamin E. Several different derivatives or forms of vitamin E are well known, for example 2,2,5,7,8-pentamethyl-6-hydroxychromane (PMC), troglitazone, Trolox C, α-tocopherol, γ-tocopherol, δ-tocopherol, β-tocopherol, α-tocotrienol, γ-tocotrienol, δ-tocotrienol, β-tocotrienol, tocopheryl acetate or tocopheryl succinate. In one particular embodiment, the vitamin E derivative is tocopheryl acetate. In one particular embodiment, the vitamin E or vitamin E analog is present in a concentration of approximately 0.001 to 2% (w/vol), for example a concentration of approximately 0.01 to 1%, or approximately 0.5%.

The term “lipidic formulation” refers to a formulation comprising one or more lipid components (fatty acids, glycerides/triglycerides, lipophilic surfactants, oils), preferably an oil or a combination of oils. The composition can therefore comprise one or more oils, for example oils of animal or plant origin, in particular palm oil, soybean oil, rapeseed oil, sunflower oil, olive oil or primrose oil. In one particular embodiment, the oil(s) is (are) present in a concentration of approximately 70 to 95% (vol/vol) or 80 to 95%, for example approximately 85 to 95%, and more particularly approximately 90%. In one particular embodiment, the composition comprises soybean oil, sunflower oil or primrose oil, preferably soybean or sunflower oil.

Biodegradable material(s) (e.g., polymer(s)) that may be used in the biodegradable microspheres are well known in the art. Examples of biodegradable materials for biodegradable microspheres include, but are not limited to, biodegradable polymers/co-polymer such as polylactide, poly(lactic acid-co-glycolic acid), poly(dioxanone), poly(trimethylene carbonate) copolymer, poly(caprolactone) homopolymer, polyanhydride, polyorthoester, polyphosphazene, poly(caprolactone) copolymer, any polymeric substances based on polylactide (PLA), polyglycolide (PGA), polycaprolactone (PCL), Poly(ethylene oxide), Poly-alginate, PEO, Poly((lactide-co-ethyleneglycoty-co-ethyloxyphosphate), Poly(LAEG-EOP), Poly(1,4-bis(hydroxyethyl)terephthalate-co-ethyloxyphosphate), Poly(BHET-EOP), Poly(1,4-bis(hydroxyethyl)terephthalate-α/t-ethyloxyphosphate)-co-1,4-bis(hydroxyethyl)terephthalate-co-terephthalate), Poly(BHET-EOP/TC, 80/20), PMMA (Polymethylmethacrylate), poly(saccharide) and proteins (hydrogel-forming proteins/polysaccharides, including whey proteins and soy proteins), e.g., starch (n-octenylsuccinate-derivatized (n-OSA) starches, maltodextrin), cellulose, chitosan (e.g., chitosan hydrolysate), xanthan gum, dextran, hyaluronate, collagen, albumin, gelatin, elastin, silk fibroin, alginate, pectin, heparin sulfate or any mixtures/combinations thereof.

In an embodiment, the biodegradable microspheres comprise casein, sodium caseinate, or calcium caseinate, preferably sodium caseinate.

In an embodiment, the biodegradable microspheres further comprise chitosan (e.g., chitosan hydrolysate), xanthan gum and/or maltodextrin.

According to one particular embodiment, the biodegradable material/polymer (e.g., sodium caseinate) is present in a concentration of approximately 0.1 to 5%, for example approximately 0.5 or 1% to 3%, and more particularly approximately 1.5%, by weight of the formulation.

The microspheres can be prepared by a variety of methods known in the art, including a single oil-in-water emulsion, a double oil-in-water emulsion, solvent evaporation, spray drying (atomisation) or coacervation of the polymer solution (See, for example, U.S. Pat. Nos. 4,652,441; 4,711,782; 4,917,893; 5,061,492; 5,407,609; 5,478,564; 5,556,642; 5,631,021; 5,643,506; 5,945,126; 5,989,463; 6,224,794; 6,270,802; 6,403,114, Jyothi et al., The Internet Journal of Nanotechnology, Volume 3 Number 1; and Nissim Garti & D. Julian McClements, “Encapsulation technologies and delivery systems for food ingredients and nutraceuticals, 2012, Woodhead Publishing Limited).

The microspheres may display desirable release kinetics, i.e., low initial burst and controlled release of the progestogen (e.g., altrenogest), over time.

The composition may comprise one or more additional ingredients (excipients or vehicles). The term “excipient” refers to any component of the composition that is not the active ingredient, and that essentially serves to improve the appearance or taste, ensure preservation, and facilitate shaping and administration of the composition. The excipient can also be used to convey the active substance to its action site and control its absorption by the body. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. See, for example, Remington's Pharmaceutical Sciences, 18^{th} Edition, A. Gennaro, Ed., Mack Pub. Co. (Easton, Pa., 1990), Chapters 88-91. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

In one particular embodiment, the composition comprises one or more antioxidants or preservative agents. These agents are used to improve the stability of the active ingredient, in particular to prevent the deterioration, rancidity and/or microbiological contamination of the preparations, for example deterioration caused by the oxidation process, hydrolysis, photodegredation and/or microbiological contamination (e.g., bacterial contamination). In one particular embodiment, the antioxidant or preservative agent(s) is (are) present at a concentration of approximately 0.001 to 5% (w/vol), for example 0.001 to 1%.

In one particular embodiment, the composition comprises one or more of the following antioxidant or preservative agents: sorbic acid, butylated hydroxyanisole and/or butylated hydroxytoluene (BHT). In another particular aspect, the composition comprises sorbic acid, butylated hydroxyanisole and butylated hydroxytoluene (BHT).

The composition may also comprise one or more alcohols. In one particular embodiment, the alcohol(s) is (are) present at a concentration of approximately 0.01 to 5% (vol/vol), for example approximately 0.1 or 0.5 to 2%, and more particularly approximately 1%. In one particular embodiment, the f composition comprises butyl alcohol.

The composition may also comprise one or more amino acids, for example L-arginine.

The composition may be in different forms/systems, for example in a capsule, a cutaneous/transdermal patch, or mixed with food. In one particular aspect, the composition is in a form allowing the controlled release of the progestogen, preferably altrenogest. In a particular embodiment, the present invention provides a cutaneous/transdermal patch comprising the formulation described herein.

The development of suitable dosing and treatment regimens for using the compositions described herein in a variety of treatment regimens, including e.g., oral, parenteral, intravenous, intranasal, intradermal, subcutaneous and intramuscular administration and formulation, is well known in the art. The amount of progestogen contained within the composition depends upon the site of administration/implantation, the rate and expected duration of release, the weight of the animal, and the nature of the condition to be treated or prevented. In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet. The progestogen may even be incorporated with excipients and used in the form of ingestible tablets, buccal tables, troches, capsules, elixirs, suspensions, syrups, wafers, and the like (see, for example, Mathiowitz et al, Nature 386:410-4, 1997; Hwang et al, Crit Rev Ther Drug Carrier Systl 5:243-84, 1998; U.S. Pat. Nos. 5,641,515; 5,580,579 and 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the progestogen may be incorporated into sustained-release or controlled-release preparation and formulations.

In an embodiment, the above-defined microspheres are formulated in a liquid formulation, e.g, in an oil or aqueous (water) solution. In another embodiment, the above-defined microspheres are formulated in a solid formulation, as dry powder.

In another aspect, the present invention relates to a method for improving the regularity of the appearance of estrus in an animal, the method comprising the administration of the formulation as defined herein to the animal.

In another aspect, the present invention relates to the use of the formulation as defined herein to improve the regularity of the appearance of estrus in an animal.

In another aspect, the present invention relates to the use of the formulation as defined herein for the preparation of a medicament to improve the regularity of the appearance of estrus in an animal.

In another aspect, the present invention relates to the use of the formulation as defined herein to delay farrowing by several days for zootechnical purposes (to have plumper products), therapeutic and breeding regime purposes.

In one particular embodiment, the animal is a farm animal, such as a cow, heifer, sow, gilt, mare, or the like.

### MODE(S) FOR CARRYING OUT THE INVENTION

The present invention is illustrated in further details by the following non-limiting examples.

### Example 1: Materials and Methods

Table 1 shows the preparation of a representative formulation according to the invention (ALTERVITAMIN, ALT).

TABLE 1

| | Quantity (for 1 L of | |
|---|---|---|
| Ingredient | formulation) | |
| Sorbic acid | 1.5 | gr |
| Butylated hydroxyanisole (BHA) | 0.07 | gr |
| Butylic alcohol | 10 | ml |
| Butylated Hydroxytoluene (BHT) | 0.07 | gr |
| Vitamin E | 0.05% | |
| Altrenogest | 2.2 | gr |

| | |
|---|---|
| Sodium Caseinate (microsphere coating) | 1.5% |

Solution A:

In a 2 L Erlenmeyer in which a magnetic agitator was positioned, the butyl alcohol was poured under agitation at 300 RPM. The calculated quantities of altrenogest, sorbic acid, butylated hydroxyanisole (BHA), butylated hydroxytoluene (BHT), and vitamin E (tocopherol acetate) were added and the mixture was agitated for 24 hours at room temperature.

Solution B:

Soybean or sunflower oil.

Solution A was mixed with solution B in a ratio of 1:10, and the mixture was agitated for 24 hours. The solution was distributed in 1 L bottles and kept at room temperature and out of direct light. The solution was then coated with the sodium caseinate to obtain microspheres using the following methods:

Method 1: Dry Microcapsule Obtained by Atomisation (Spray-Drying)

1.5 g Sodium caseinate

15 g sunflower or soybean oil with Altrenogest (see above)

13 g maltodextrin 10

70.75 g distilled water

The emulsion comprising the ingredients above was pre-homogenized with a Polytron™ homogenizer for 3 min at 9000 rpm, then homogenized with a two-stage Avestin™ homogenizer (3*5000 psi+1*500 psi).

Spray drying was performed under the following conditions: spray dryer BUCHI® Mini Spray Drier B-191 with a 2 mm nozzle diameter, air pressure of 60 psi. The emulsion was fed into the main chamber through peristatic pump, feed flow rate was 400 ml/h, inlet air temperature was 160° C. and outlet temperature was 65° C. The dried microcapsules were collected in a cyclone.

Particle size distribution of the final emulsion was less than 1 micron and the powder obtained by atomisation or freeze drying had good rehydration properties, low surface oil and low oxidation during the powder storage. Furthermore, the emulsion in liquid form containing 1.5% sodium caseinate and 15% sunflower oil was physically stable when pasteurized with no preservation agents added to it. Maltodextrin was used as a “wall material” to permit further drying of the emulsion. Additionally, the retention of Altrenogest was well preserved in both the liquid and dry form for a period of at least 5 months at ambient temperature.

Method 2: Microcapsules Prepared by a Coacervation Process (Multilayered Emulsion)

A 1.5% sodium caseinate (Na-caseinate) solution was prepared by mixing 501 g of bi-distilled water with 9 g of Na-caseinate (whey protein isolate could also be used). The pH was adjusted to 3.5, and 90 g of sunflower or soybean oil with Altrenogest (see above) was added to the mixture. The mixture was pre-homogenized with a Polytron™ homogenizer for 3 min at 9000 rpm, then homogenized with a two-stage homogenizer Avestin™ (3*5000 psi+1*500 psi).

A solution of 20% arabic gum (acacia) was prepared by mixing 320 g of bi-distilled water with 80 g arabic gum. The solution was mixed with a Polytron™ homogenizer, deaerated and the pH was adjusted to 3.5.

A solution of chitosan hydrolysate (food grade hydrolysate) was prepared by mixing 12 g of chitosan with 588 g of bi-distilled water. The solution was mixed with a Polytron™ homogenizer, deaerated and the pH was adjusted to 3.5.

367.5 g of the arabic gum solution and 525 g of the chitosan solution were mixed with a magnetic bar.

The coacervate was prepared by combining 107.5 g of bi-distilled water, 500 g of the Na-caseinate emulsion and the arabic gum-chitosan solution, and adjusting the final pH to 3.5.

This solution may be used either as liquid or dry powder.

Animals and Artificial Insemination

The tests took place in 9 livestock farms with 8-13 sows per farm and a total of 175 animals (Table 2).

TABLE 2
Dose and duration of treatment of sows
according to the farming group.

| | | | | | Duration | |
|---|---|---|---|---|---|---|
| | | | | | of treat- | Insemi- |
| | | Sows | | Dose | ment | nation |
| | | number | | (mg/ml/d) | (d) | (day) |
| Group 1 | Livestock 1 | 175 | | 2 - ALT | 15r | Simple at d |
| | | | | | | 4 post-estrus |
| | Control 1 | 175 | | 2.2 - REG | 15r | Simple at d |
| | | | | | | 4 post-estrus |
| Group 2 | Livestock 2 | Lot-1 | 10 | 2 - ALT | 10 | Double at |
| | | | | | | estrus |
| | | Lot 2 | 10 | 4 - ALT | 15r | Double at |
| | | | | | | estrus |

| | | | | |
|---|---|---|---|---|
| Control 2 | 20 | 2.2 - REG | 15 | — |

REG = Regumate ® (oral oil solution of altrenogest 2.2 mg/ml (0.22%))
ALT = oral oil solution of altrenogest microcapsules prepared according to method 2 above

Sows of livestock that have undergone hormonal therapy have all been systematically inseminated from the fourth day of the estrus appearance. However, for livestock 2, double insemination took place at the day of induced estrus. The number of spermatozoa used per insemination was 8×10⁹.

Monitoring

At the time of farrowing, number of piglets born and stillborn was controlled. Differences between treatments were tested by variance test analysis or ×2 test.

### Example 2: Results

Estrus Appearance

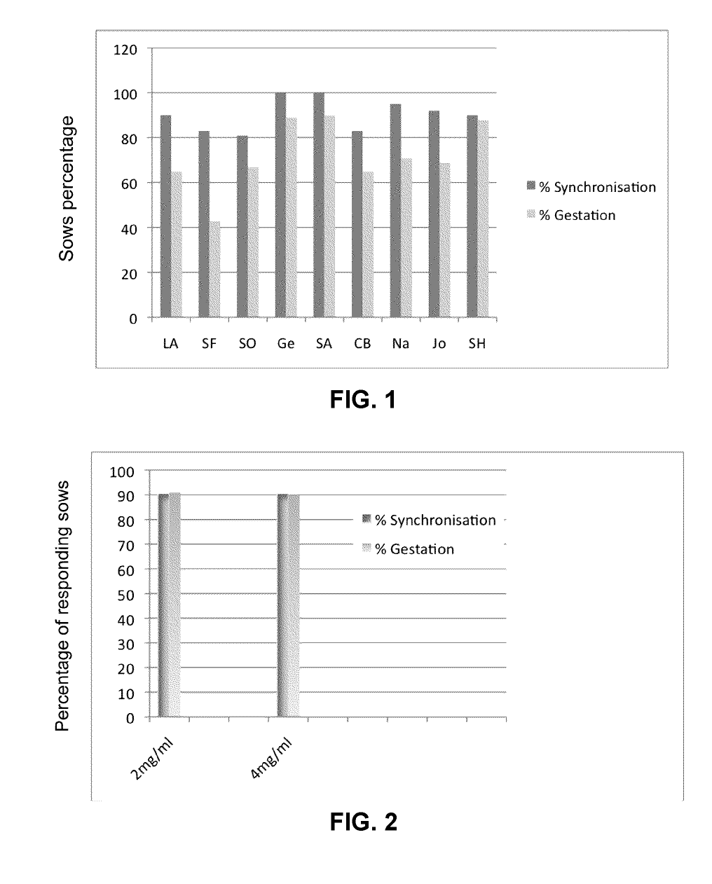

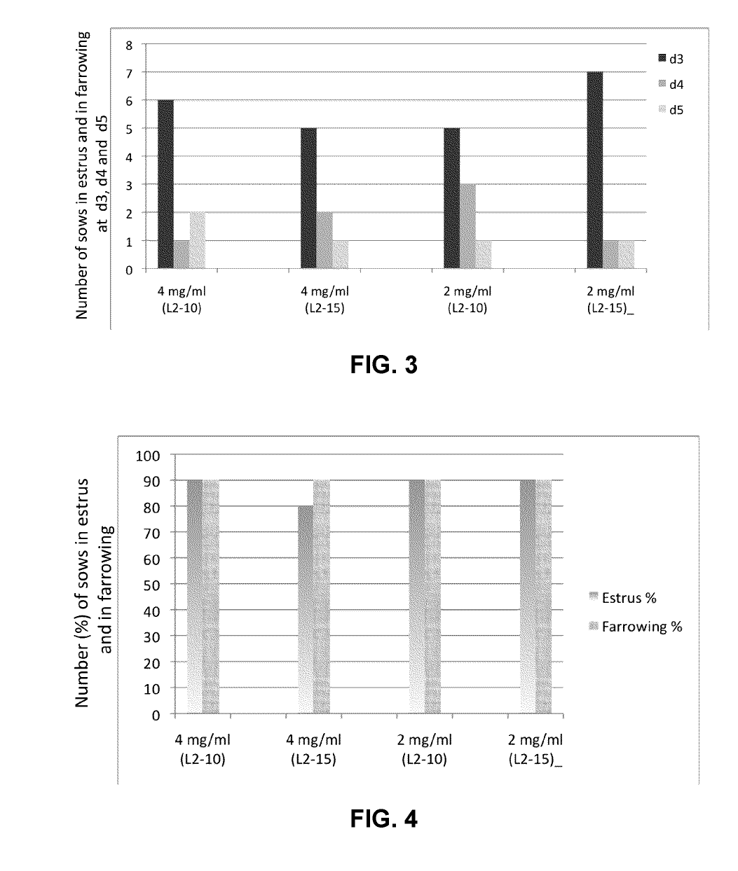

The estrus occurs between 1 and 7 days after treatment in over 90% of the treated females (FIGS. 1 and 2). The maximum of sows that came in estrus (66%) occurred at d3 post-treatment. FIG. 1 shows that the rate of synchronized sows in the nine farms studied is 81% to 100% with an average rate of 90.4%. From these sows that came to estrus, gestation rate is 43-90% with an average rate of 71.9%. There is no significant influence of the ALT dose used (2 mg/ml vs. 4 mg/ml) on the percentage of sows that came in estrus (FIGS. 2 and 3). Both treatment groups (10 days and 15 days) were comparable, including the proportion of females cycled before treatment. Thus, over 175 animals that have undergone hormonal treatment, only 10 had not estrus behavior between J3 and J5 and returned later to estrus. In animals not treated with ALT, the percentage of sows that did not come to estrus after 10 days varies considerably between farms: 11% for livestock 2 and 9% for livestock 1. However, all sows have been covered in the months after weaning.

Farrowing and Prolificacy Rates

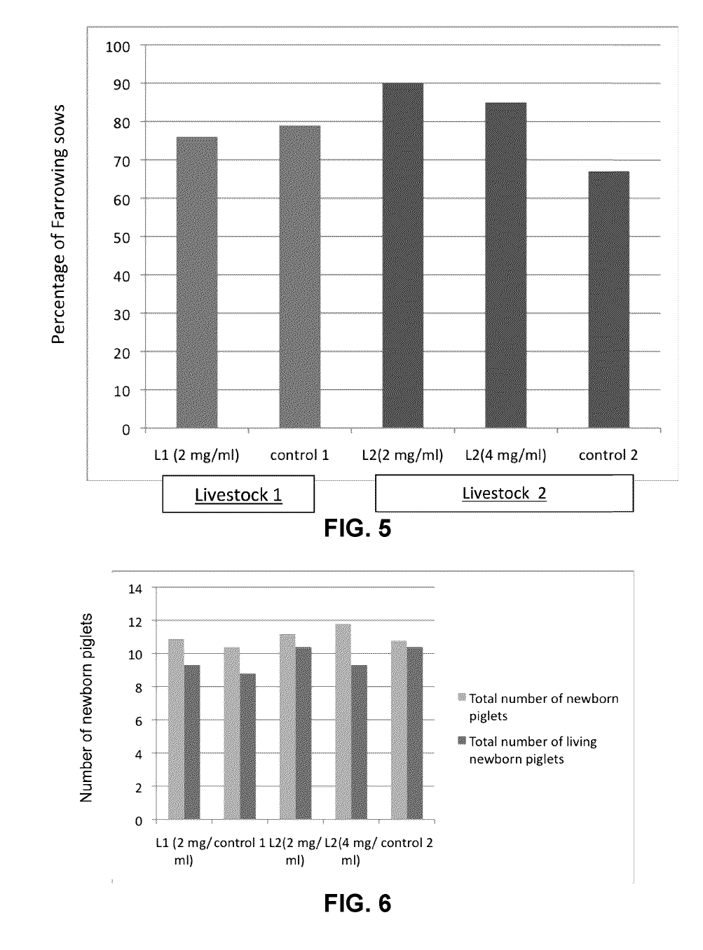

Systematic insemination twice of all sows treated with ALT and REG (Livestock 1) on day d4 and d5 was followed by farrowing in 71.8% of cases. When the double insemination was performed at induced estrus (Livestock 2), the farrowing rate was 90% for both ALT doses used (2 and 4 mg/ml) (FIG. 4) (88% in untreated sows: no significant difference). The gestation rate was higher in the group of sows treated for 15 days (70.1%) than in the group treated for 10 days (62.6%): lengthening the duration of ALT treatment has improved significantly fertility at induced estrus. In the case where sows are inseminated systematically at day d4 and d5, only one female on six was found pregnant without having estrus behavior at the time of insemination. In livestock 1, the interval weaning-fertilizing protrusion is 25.2 days for the treated group and 19.2 days for the control group. In livestock 2, it is 11.1 days and 15.6 days for the treated group and 7.6 days for the control group. There is thus a tendency for duration lengthening when sows underwent the ALT treatment. Depending on the dose of ALT used, prolificacy varies but none of the differences was significant. However, in both cases, the treated pigs at a dose of 4 mg/ml of ALT, have a significantly higher average number of piglets (Livestock-1: 10.6 and Livestock-2: 11.8) than sows that received 2 mg/mL (Livestock 2: 11.2) (FIGS. 5 and 6). The treatment of sows with a dose of 2 mg/ml of ALT gives farrowing rates comparable (Livestock-1: 76%) or even significantly higher (Livestock-2: 90%) than conventional treatment with a dose of 2.2 mg/ml REG in the control group (control 1: 79% and control 2: 67%).

The use of ALT for estrus synchronization allows a good grouping of inseminations because, over a period of 72 hours, 90% of sows are in heat. Similar figures were cited earlier, when they are treated with a mixture of androgen and estrogen. However, some farms as Livestock-2 record rates of sows in estrus in the same levels when the animals are not treated. These figures may be rather exceptional, indicating significant variations between farms for the percentage of non-in estrus sows after 7 days (0-46%). A double insemination made according to early induced-estrus appearance gives better results than two inseminations at predetermined day, but the differences may be due to factors other than the timing of artificial insemination. Estrus synchronization rate obtained in the studies described herein was about 90%, which is higher than that obtained in most studies using progestogens. This high rate can be explained in part by the presence of an antioxidant and/or of a coating to ensure the bioavailability and chemical stability of the ingredients, for example against animal digestive enzymes. Finally, the good rate of cyclicity influences the synchronization rate before treatment (88.0%).

In conclusion, these results show that the experimental treatment with ALT has an efficacy that is at least equivalent or even superior (at least in terms of estrus synchronization and/or farrowing rates) to conventional treatment with Regumate® on the species type of studied animals.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A biodegradable microsphere composition comprising (a) a biodegradable microsphere material; and (b) a lipidic liquid formulation comprising (i) an effective amount of a progestogen, and (ii) vitamin E or an analog of vitamin E, incorporated within said biodegradable material, into said microspheres.

2. The biodegradable microsphere composition according to claim 1, wherein the progestogen is altrenogest:

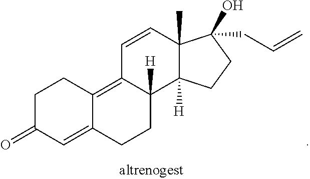

3. The biodegradable microsphere composition of claim 1, comprising an analog of vitamin E, wherein said analog of vitamin E is tocopheryl acetate.

4. (canceled)

5. The biodegradable microsphere composition of claim 1, comprising one or more antioxidants or preservative agents.

6. The biodegradable microsphere composition of claim 5, wherein the antioxidant or preservative agent(s) is (are) sorbic acid, butylated hydroxyanisole and/or butylated hydroxytoluene (BHT)

7. The biodegradable microsphere composition of claim 5, wherein the antioxidant or preservative agent(s) is (are) present in the formulation at a concentration of about 0.001 to 1% or about 0.01% to 0.02%.

8. (canceled)

9. The biodegradable microsphere composition of claim 7, comprising about 0.001% to 0.01% of butylated hydroxyanisole, about 0.001% to 0.01% of butylated hydroxytoluene, and about 0.01 to 0.02% of sorbic acid.

10. The biodegradable microsphere composition of claim 1, further comprising an alcohol.

11. (canceled)

12. The biodegradable microsphere composition of claim 10, wherein the alcohol is present at a concentration of about 0.5 to 2% or about 1%.

13. (canceled)

14. The biodegradable microsphere composition of claim 1, comprising an oil.

15. (canceled)

16. The biodegradable microsphere composition of claim 14, wherein the oil is present at a concentration of approximately 85% to 90%.

17. The biodegradable microsphere composition of claim 1, wherein the progestogen is present at a concentration of about 0.01 to 1% or about 1%.

18. (canceled)

19. The biodegradable microsphere composition of claim 1, wherein the vitamin E or the vitamin E analog is present at a concentration of approximately 0.01 to 1% or 0.05%.

20. (canceled)

21. The biodegradable microsphere composition of claim 1, wherein the biodegradable microsphere material comprises a biodegradable polymer, a polysaccharide, a protein, or any mixtures thereof.

22-23. (canceled)

24. The biodegradable microsphere composition of claim 21, wherein the biodegradable microsphere material comprises xantham gum, chitosan and/or maltodextrin.

25. The biodegradable microsphere composition of claim 1, which is in liquid or solid form.

26. The biodegradable microsphere composition of claim 25, wherein (i) the biodegradable microsphere composition is in liquid form and the biodegradable microspheres are in an oil or aqueous solution; or (ii) the biodegradable microsphere composition is in solid form and the biodegradable microspheres are in a dry powder.

27-28. (canceled)

29. The biodegradable microsphere composition of claim 1, wherein the biodegradable microsphere composition is in a capsule, a transdermal patch or a food product.

30. A method for (i) improving the regularity of the appearance of estrus and/or (ii) synchronizing time of insemination in an animal, the method comprising the administration of the biodegradable microsphere composition of claim 1 to the animal.

31-32. (canceled)

33. The method of claim 30, wherein the animal is a sow or gilt.

34-39. (canceled)
